(19) United States
(12) Patent Application Publication
(10) Pub. No.: US 2020/0281572 A1
(43) Pub. Date: Sep. 10, 2020

(54) A SELF-SAMPLING DEVICE FOR VAGINAL FLUID COLLECTION

(71) Applicant: Aprovix AB, Uppsala (SE)

(72) Inventors: Henry Andersson, Uppsala (SE); Sören Nygren, Åkersberga (SE); Erik Wilander, Tällberg (SE)

(73) Assignee: Aprovix AB, Uppsala (SE)

(21) Appl. No.: 16/492,369

(22) PCT Filed: Mar. 9, 2018
(86) PCT No.: PCT/EP2018/055918

§ 371 Date: Sep. 9, 2019

(30) Foreign Application Priority Data
Mar. 10, 2017 (SE) 1750273-3

(51) Int. Cl. A61B 10/00

(52) U.S. Cl. CPC A61B 10/0045; A61B 2010/0074; A61B 2010/0003

(57) ABSTRACT

A self-sampling device (1) for vaginal fluid from a vagina of a user, said self-sampling device (1) comprising an elongate first body (2) for insertion into the vagina of the user, and a sample collection body (3) for collecting a vaginal fluid sample from the user. The sample collection body (3) is attachable to an inner end portion (4) of the elongate first body (2) by a releasable connection means (5) comprising a shaft, a hole or recess for receiving the shaft, and a snap-locking means. The self-sampling device is used to take a vaginal fluid sample intended for subsequent laboratory analysis, for example, but not limited to, DNA analysis for HPV.

### TECHNICAL FIELD

The present invention relates generally to the field of screening and health control. More specifically the invention relates to self-sampling devices for vaginal fluid testing to detect for example virus-associated cervical cancer, microbial infections and pathological changes.

### BACKGROUND

Pap smear, cervical smear, or smear test are different names of a method of cervical screening used to detect potentially pre-cancerous and cancerous processes in the cervix. Abnormal findings are often followed up by more sensitive diagnostic procedures, and, if warranted, interventions that aim to prevent progression to cervical cancer. A common type of laboratory analysis made on sample material taken from the vagina is DNA analysis for HPV (human papillomavirus). Traditionally, cervical smear testing requires scraping of a woman's cervix with a sampling device, such as a spatula or a brush. This sampling is generally performed by medical professionals like gynecologists, midwifes or nurses in a clinical environment. Many women, who now refrain from such gynecological testing, would participate if the sampling could be carried out at home and/or by the women themselves. Self and home sampling would therefore increase the participation in the screening, and by that means, decrease the incidence of cervical cancer. US 2003/0028123 A1 discloses an intra-vaginal self-administered cell collection device, but no snap-fit connection. U.S. Pat. No. 5,445,164 A discloses a quick-release connection in the context of a cervical tissue sampling device, but the release mechanism is based on sliding a sleeve rearward along shaft until engaged tab and portions are exposed. The pin may then be manually disengaged from aperture to complete detachment of stem from shaft. In addition to the above, sampling systems are also in demand for DNA analysis. Law enforcement officials, paternity agents, etc. are constantly taking DNA samples to help solve crimes, determine paternity, etc. As the results of the tests done on these samples dramatically affect people's lives and may be desired as evidence in legal proceedings, the sampling must be done in a manner in which the sample contamination is reduced or avoided.

One example of a system suitable for self-testing is described in European Patent EP1903946B1 to Aprovix AB, Sweden. This known system brings many advantages over prior art solutions.

The sample collection element is removably connectable with the shaft by a snap-fit connection which is formed by protrusions at an end of the sample collection means and adapted to snap-fit in grooves contained in extensions at the other end of the shaft. The snap-fit connection with protrusions and grooves is dependent on exact fit between protrusions and grooves in order to perform consistently for holding the sample collection element connected to the handle and also allow for detachment of the sample collection element from the handle when the sample collection element is within the sealable unit. Such exact fit requires extensive quality systems with measurement of produced parts (i.e. handle and sample collection element). Testing is time-consuming and expensive and so is discarding batches of produced parts.

During sampling with the self-sampling device shown in EP1903946B1 there is a slight risk that excessive side forces brings the snap locking engagement between sample collection body and handle out of engagement, resulting in losing control of the sample collection body at sampling. Further, the protrusions are released from the grooves contained in the extension by rotation of the sample collection element. However, the protrusions often tend to grip the recesses also after rotation, at least to some extent, wherein easy disengagement is prevented.

Hence, there is the need of an improved self-sampling device performing consistently without requiring extensive quality testing in production.

### SUMMARY

An object of the invention is to provide an improved self-sampling device which mitigates the drawbacks discussed above.

According to a first aspect of the invention, this and other objects are achieved by a self-sampling device for vaginal fluid from a vagina of a user, said self-sampling device comprising an elongate first body for insertion into the vagina of the user, and a sample collection body for collecting a vaginal fluid sample from the user. The sample collection body is attachable to an inner end portion of the elongate first body by a releasable connection means. The elongate first body is configured such that it is operable by the user to collect a fluid sample from the vagina with the sample collection body attached to the first body. The releasable connection means comprises a first interlocking means provided on one of the first body and the sample collection body, and a second interlocking means provided on the other one of the elongate first body and the sample collection body. The first interlocking means is provided with a shaft defining a rotational axis substantially transversal to a longitudinal axis of the first body. The second interlocking means comprises a first hole or recess configured to receive the shaft upon connection of the first and second interlocking means for rotation of the sample collection body relative to the first body about the rotational axis between a first position, in which the longitudinal axis of the sample collection body is aligned with the longitudinal axis of the first body, and a second position in which the longitudinal axis of the sample collection body is rotated about the rotational axis away from said first position a predetermined distance. The first and second interlocking means are further provided with first and second respective corresponding snap-locking means configured to releasably engage each other when in the first position to prevent relative rotation between the first and second interlocking means when the snap-locking means are engaged. The first and second interlocking means are configured such that in said second position the first and second interlocking means are disengageable by movement of the shaft out of the first hole or recess in a predetermined disengagement direction. Also, the first and second interlocking means are configured such that in said first position the shaft is confined within said first hole or recess.

The shaft and corresponding hole or recess provides for well-defined relative rotational movement between sample collection body and first body. The snap-locking means functions to hold the sample collection body in the first position during use, enabling controlled movement of the sample collection body by holding an outer end portion of the first body and operating the sample collection body for sampling, just like the use described in EP1903946B1 mentioned above. The shaft of the first attachment means limits the degrees of freedom available for the sample collection body to move, so that it can only move from its first position according to said well-defined rotational movement. Thus, the snap-locking means needs mainly to cope with momentum applied to the sample collection body and needs not to cope with relative translational movement. Such combination of shaft and snap-locking means provides for lower risk of losing control of the sample collection body at sampling. The design even enables use of a weaker snap-lock mechanism with sustained control of the sample collection body in the vagina. Using a weaker mechanism provides for use of less material and thereby less environmental impact. After sampling, the sample collection body is withdrawn from the vagina and inserted into a small container for protection of the sample. After insertion, momentum is applied to rotate the sample collection body and first body into the second position. In said second position, the first body is simply moved away from the sample collection body such that the sample collection body is left in the container by gravitational forces acting on the sample collection body. The first body is then discarded and the container sealed, for example by a lid, ready for future analysis.

Since the first and second interlocking means are disengageable in said second position, but not in said first position, a firm hold is provided in the first position, and easy disengagement is provided in said second position. The sample collection body can easily be separated from said handle with a minimum of force and without need of manually holding the sample collection body to pull it off the first body. Also, the sample collection body is well controlled during sampling.

In an embodiment, the shaft is provided at a first proximal portion of the first interlocking means. Further, the first one of the corresponding snap-locking means is provided at a first distal portion of the first interlocking means.

The proximal portion of the respective interlocking means is the portion that is closest to the sample collection body or first body on which it is provided. By providing the shaft at the proximal portion of the first attachment means, there is little distance between shaft and the sample collection body or first body on which it is provided, and hence the shaft is rigidly attached and cannot move much. This provides for better control of the relative rotation/movement between the first body and the sample collection body. Likewise, the distal positioning of the first snap-locking means provides for a greater distance between snap-locking means and the sample collection body or first body to which the first interlocking means is attached, and thus enables increased flexibility of the snap-locking means. Increased flexibility allows for design of a snap-locking mechanism where a main source of biasing force is the first interlocking means itself, thereby providing for a simple and robust design.

To further specify the location of the proximal and distal portions, the first proximal portion of the first interlocking means is adjacent the first body or sample collection body on which the first interlocking means is provided. Likewise, the first distal portion is further away from the sample collection body or first body on which the first interlocking means is provided than the first proximal portion.

In an embodiment, the first hole or recess is laterally open such that the shaft is moveable laterally out of the first hole or recess from said second position at disengagement.

Making the first hole or recess laterally open enables the shaft to be moved laterally/radially out of the first hole or recess rather than having to be moved along the longitudinal axis of the shaft.

In an embodiment, the first interlocking means comprises a forked body comprising opposite shanks together defining an intermediate space extending along the longitudinal axis of the first body. The shaft extends from one shank to the other across said intermediate space. The second interlocking means comprises an elongate stem adapted to fit in the intermediate space between the shanks. Also, the first hole or recess of the second interlocking means is laterally open for receiving the shaft.

Making the first hole or recess laterally open enables the shaft to be moved laterally/radially out of the first hole or recess rather than having to be moved along the longitudinal axis of the shaft. This in turn allows for the shaft to be fixed at both ends without hindering disengagement of the first and second interlocking means. Fixing the shaft at both ends provides a robust fixation of the shaft, thereby enabling use of weaker shaft with less material. Using less material is of advantage, since the self-sampling device is a single-use device and thus cost and environmental impact is of more importance that long-term durability.

In an embodiment, the first hole or recess is laterally open in a direction along the length of the second interlocking means.

Such configuration of the first hole or recess enables easy disengagement of the sample collection body once it is positioned vertically in the container and the first member rotated to bring the sample collection body to the second position. Since the shaft is moveable vertically out of the first hole or recess and the sample collection body is thus free to fall down into the container by the force of gravity.

In an embodiment, the shaft is mainly cylindrical with at least one recessed portion along the length of the shaft. Also, the first hole or recess has a cross sectional shape with a circular mid portion corresponding to the main diameter of the shaft. Said mid portion opens laterally outwards in the form of a passage with a smallest width less than the diameter of the shaft. Further, the recessed portion(s) of the shaft defines a projected profile of the shaft in a plane through the longitudinal axis of the shaft, wherein said projected profile is smaller than the passage such that the shaft is movable through the passage from said second position.

By providing the shaft with such recessed portions, the shaft is prevented from moving through the path except for when the shaft is oriented in the second position in which the shaft exposes its projected profile smaller than the passage. This allows the first and second interlocking means to engage to always allow relative rotation whilst at the same time preventing relative translation when not in said second position, thereby confining the shaft within the first hole or recess.

In an embodiment, the passage widens outwardly away from the circular mid portion. Such widening configuration of the passage provides surfaces for guiding the shaft into the passage at assembly of the shaft. Thereby making both manual and automatic assembly easier and more predictive.

In an embodiment, the first hole or recess comprises an auxiliary recessed portion extending inwards from the circular mid portion along the length of the second interlocking means, away from the passage and partly towards the second snap-locking means. The width of the auxiliary recessed portion is less than the diameter of the mid portion.

The auxiliary recessed portion extending further inwards improves the resiliency of the second connection means such that the shaft can be forced into the circular mid portion even though not in said second position. This in turn enables quick and easy assembly of first body and the sample collection body. Another advantage is that the improved resiliency enables controlled biasing between the shaft and the first hole or recess, should a biasing engagement be of interest.

In an embodiment, the corresponding snap-locking means are provided in the interface between stem and shanks.

Providing the snap-locking means in the interface between shanks and stem enables the resilience of the shanks to force the corresponding snap-locking means together, such as by forcing one or more protrusions into one or more corresponding recesses. This provides an easy means of achieving a desired biasing functionality of the snap-locking means.

In an embodiment, protrusions of the first snap-locking means are provided on the shanks. Further, the at least one recess of the snap-locking means is provided in the stem. Also, the protrusions of the first snap-locking means comprise forwardly chamfered portions oriented for the stem to gradually push the protrusions apart upon insertion of the stem between the chamfered portions along the longitudinal axis of the first body.

This configuration of the snap-locking means promotes quick and easy assembly of first body at insertion of the stem along the longitudinal direction of the first body into the intermediate space, wherein the risk of damaging the protrusions during assembly is lowered since the chamfered portions make it easier for the stem to push apart the protrusions of the snap-locking means during assembly and thereby reduces the stress on the protrusions. Deformed or damaged surfaces on the protrusions of the snap-locking means would increase the risk of malfunction of the self-sampling device, such as too loose snap-functionality or direct risk of losing control of the sample collection body at sampling.

Table of reference numerals

| | |
|---|---|
| 1 | self-sampling device |
| 2 | first body |
| 3 | sample collection body |
| 4 | inner end portion |
| 5 | releasable connection means |
| 6 | first interlocking means |
| 7 | second interlocking means |
| 8 | shaft |
| 9 | rotational axis |
| 10 | longitudinal axis of first body |
| 11 | first hole or recess |
| 12 | first snap-locking means |
| 13 | second snap-locking means |
| 14 | first proximal portion |
| 15 | first distal portion |
| 16 | first shank |
| 17 | second shank |
| 18 | longitudinal axis of sample collecting body |
| 19 | disengagement direction |
| 20 | intermediate space |
| 21 | stem |
| 22 | passage |
| 23 | mid portion |
| 24 | recessed portions |
| 25 | auxiliary portion |
| 26 | container |
| 27 | rotational axis defined by hole or recess |
| 28 | outer end portion |
| d1 | smallest width of passage |
| d2 | projected profile width of shaft |
| d3 | main diameter of shaft |
| D | predetermined distance |
| P | projection plane |

### BRIEF DESCRIPTION OF DRAWINGS

FIGS. 1-18 all show a first embodiment of the invention.

FIGS. 1-7 show views in which the sample collection body is rotated to its second position, ready for disengagement from the rest of the self-sampling device.

FIGS. 8-15 show views in which the sample collection body is rotated to its first position ready for sampling.

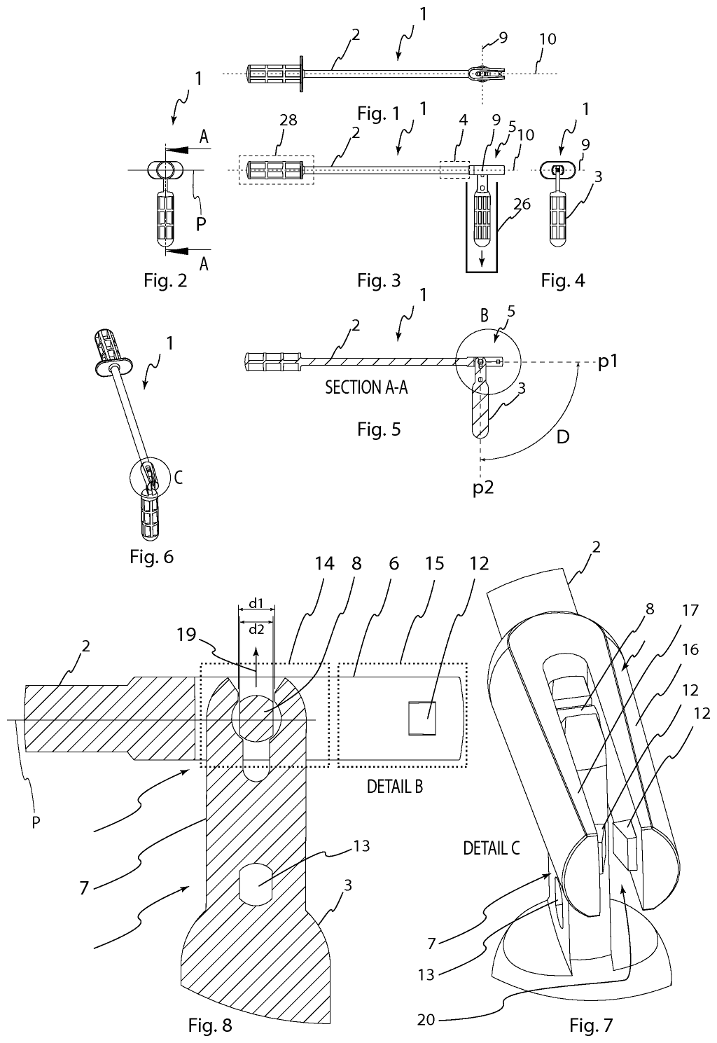

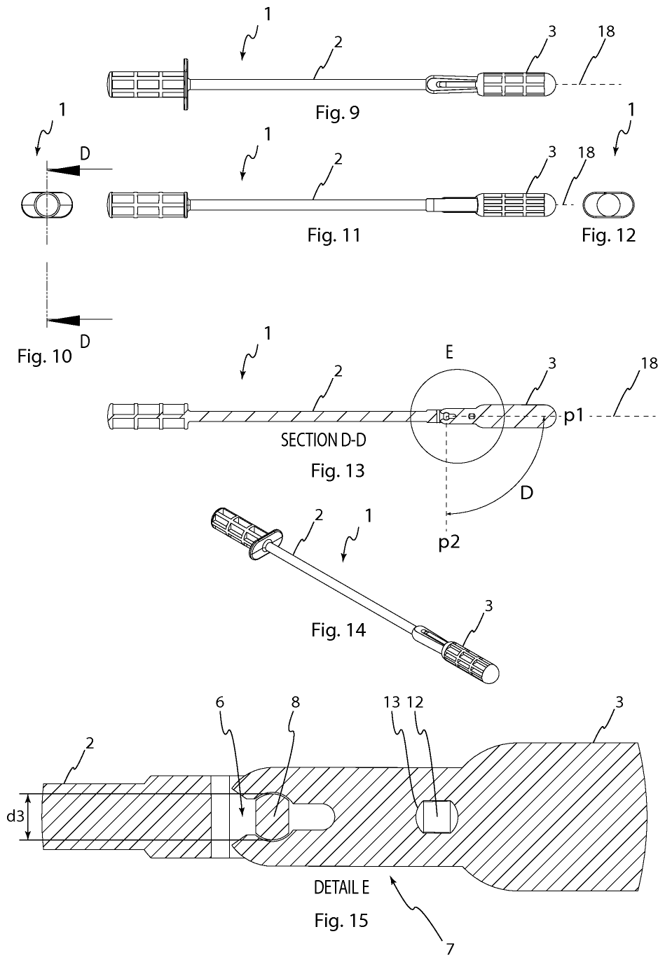

FIGS. 1 and 9 are top views.

FIGS. 2 and 10 are back views.

FIGS. 3 and 11 are side views.

FIGS. 4 and 12 are front views.

FIGS. 5 and 13 are sectional side views.

FIGS. 6 and 14 are diagonal perspective views from above.

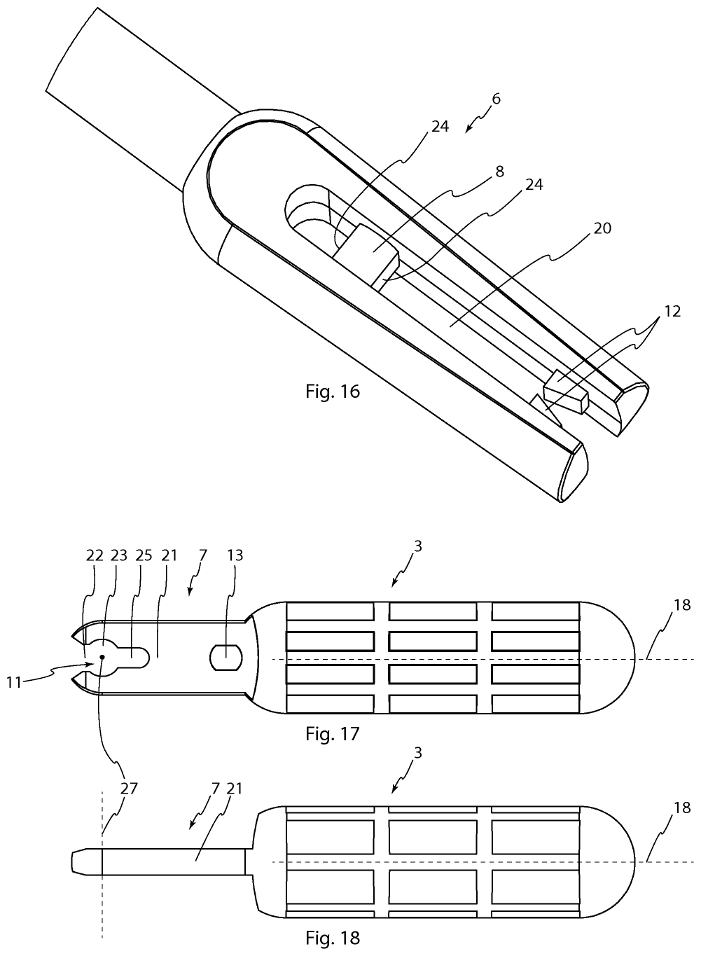

FIGS. 7, 8 and 15 are enlarged views of the first and second interlocking means.

FIG. 16 is an enlarged detail view showing the first interlocking means.

FIG. 17 is a side view of the sample collection body and the second interlocking means provided on the sample collection body.

FIG. 18 is a top view of the sample collection body and the second interlocking means provided on the sample collection body also shown in FIG. 17.

### DETAILED DESCRIPTION

The disclosed embodiments will hereinafter be described in more detail with reference to the accompanying drawings in which some embodiments of the invention are shown. This invention may, however, be embodied in many different forms and should not be construed as limited to the embodiments set forth herein. Rather, these embodiments are provided by way of example so that this disclosure will be thorough and complete, and will fully convey the scope of the invention to those skilled in the art. Like numbers refer to like elements throughout.

The self-sampling device of the present invention represents a further development of the self-sampling device described in EP1903946B1 and it is to be used in the same manner for collecting a sample and sending it for analysis by mail in a dedicated small container/sealable unit. Therefore, this type of use of the self-sampling device is known and the present description will focus on the new improved design of the self-sampling device itself. At sampling, the self-sampling device operated to rotate the sample collection body around its longitudinal axis while pushing it laterally towards the mucous membranes of the vagina such that vaginal fluid is stuck in recesses of the sample collection body.

Hence, the present invention provides a self-sampling device 1 which may be employed by an individual to easily and reliably, at home or at a visit to a medical location, under hygienic conditions, take a vaginal fluid sample intended for subsequent laboratory analysis, for example, but not limited to, DNA analysis for HPV. The samples can be transported in a small sealable container without risk of contamination or transmission of infective agents, and thereafter be analyzed by chemical methods, such as DNA analysis, or by other immunological or microbiological methods.

With reference to FIGS. 1-18, a first embodiment of the self-sampling device 1 according to the invention will be described in the following. The self-sampling device 1 comprises an elongate first body for insertion into the vagina of the user, and a sample collection body 3 for collecting a vaginal fluid sample from the user. The sample collection body 3 is attachable to an inner end portion 4 of the elongate first body by a releasable connection means 5. The inner end portion is the portion of the first body that in use is inserted into the vagina, whereas an outer end portion of the first body is a portion that in use is held by the user operating the self-sampling device. The first body is thus used as a handle.

The elongate first body is configured such that it is operable by the user to collect a vaginal fluid sample from the vagina with the sample collection body 3 attached to the first body. This implies that the first body is of suitable length and shape to be handled by an individual for performing self-sampling in the vagina. In the first embodiment, the length of the first elongate body is 145 mm wherein the narrow cylindrical body portion is 93 mm long and has a diameter of 4 mm, and wherein the gripping portion has a width/diameter of 10 mm and a length of 32 mm. The rest of the first elongate-body is occupied by a first interlocking means 6 as described below.

The releasable connection means 5 comprises a first interlocking means 6 provided on the elongate first body and a second interlocking means 7 provided on the sample collection body 3.

The first interlocking means 6 comprises a forked body comprising opposite shanks 16, 17 together defining an intermediate space 20 extending along the longitudinal axis 10 of the first body. A shaft 8 extends from one shank 16 to the other 17 across said intermediate space 20 for defining a rotational axis 9 substantially transversal to a longitudinal axis 10 of the first body. Both opposite ends of the shaft 8 are thus attached/supported.

The second interlocking means 7 comprises an elongate stem 21 adapted to fit in the intermediate space 20 between the shanks 16, 17. The stem 21 is provided with a through hole configured to receive the shaft 8 upon connection of the first 6 and second 7 interlocking means for rotation of the sample collection body 3 relative to the first body about the rotational axis 9 between a first position, in which the longitudinal axis 18 of the sample collection body 3 is aligned with the longitudinal axis of sample collection body 3 as shown in FIG. 9-15, and a second position in which the longitudinal axis 10 of the sample collection body 3 is rotated about the rotational axis 9 away from said first position a predetermined distance D, as shown in FIGS. 1-8.

The first 6 and second 7 interlocking means are further provided with first and second respective corresponding snap-locking means configured to releasably engage each other when in the first position to prevent relative rotation between the first 6 and second 7 interlocking means when the snap-locking means are engaged. The corresponding snap-locking means 12, 13 are provided in the interface between stem 21 and shanks 16, 17 with opposite protrusions 12 extending inwards into the intermediate space 20 between the shanks 16, 17, and with the stem 21 being provided with a through recess 13 corresponding to said opposite protrusions 12.

The first 6 and second 7 interlocking means are configured such that in said second position the first 6 and second 7 interlocking means are disengageable by movement of the shaft 8 out of the hole 11 in a predetermined disengagement direction 19. Also, the first 6 and second 7 interlocking means are configured such that in said first position the shaft 8 is confined within said first hole 11.

The first hole 11 of the second interlocking means 7 is laterally open in a direction along the length of the second interlocking means 7. Such configuration of the first hole or recess enables easy disengagement of the sample collection body once it is positioned vertically in the container and the first member rotated to bring the sample collection body to the second position. Since the shaft is moveable vertically out of the first hole or recess and the sample collection body is thus free to fall down into the container by the force of gravity. It should be understood that within the context of this description ‘laterally open’ means that the hole 11 is open in a direction perpendicular to a rotational axis defined by the hole 11, for example in a radial direction should the hole 11 be of cylindrical or conical shape. The rotational axis defined by the hole 11 corresponds to the rotational axis 9 defined by the shaft 8, when the first 6 and second 7 interlocking means are connected. Here it should be understood that the hole 11 could take other forms than cylindrical, as long as the hole 11 fits to the shaft 8 to support the sample element for rotation about the rotational axis 9. Generally, the hole 11 needs to exhibit at least three supporting surfaces for being distributed around the shaft 8 to control the rotational movement about the shaft 8.

The shaft 8 is mainly cylindrical with two recessed portions 24 extending along the length of the shaft 8, said portions being provided on opposite sides of the longitudinal axis of the shaft 8. The hole 11 has a cross sectional shape with a circular mid portion 23 corresponding to the main diameter of the shaft 8. As illustrated in FIG. 7, the main diameter d3 is to be understood as being the diameter of the shaft 8 without regard to the recessed portions 24. The opposite recessed portions 24 are planar, but could in other embodiments have other shapes, such as curved in one or two directions. The circular mid portion 23 could have other shapes, as long as it functions to support the second interlocking means 7 for rotation about the shaft 8.

A shown in FIGS. 7 and 15, the mid portion 23 opens outwardly in the form of a passage 22 with a smallest width d1 less than the main diameter d3 (See FIG. 15) of the shaft 8.

The recessed portions 24 of the shaft 8 define a projected profile of the shaft 8 in a plane P through the longitudinal axis of the shaft 8 normal to the disengagement direction 19. The projected profile is smaller than the passage 22 such that the shaft 8 is movable through the passage 22 from said second position, for example having a projected profile width d2 smaller than the smallest width d1 of the passage 22.

It should be understood that many shapes of the shaft 8 and of the passage 22 are possible within the scope of the present invention, and that the important teaching is that rotation of the shaft 8 relative to the hole 11 exposes differently shaped projected profiles, such that in a specific position (i.e. the second position), the projected profile fits within the bounds of a correspondingly projected profile exposed by the passage 22 of the second interlocking means 7, and such that in other positions, the projected profile of the shaft 8 does not fit within the bounds of the projected profile exposed by the passage 22. Thus, the shaft 8 is confined within the hole 11 except for in the second position where it is movable straight out of the hole 11. This enable controlled relative rotation between first 6 and second 7 interlocking means 7 within a predetermined first operational range (not in the second position, but between the second position and the first position), whilst also enabling free detachment of the first 6 and second 7 interlocking means when in another operational range outside the first operational range (i.e. in the second position). The configuration of the first embodiment depicted in FIGS. 1-15 is advantageous since it is robust and easy to produce using relatively simple injection molding tools.

The passage 22 widens outwardly away from the circular mid portion 23. This makes it easier to move the shaft 8 into the passage 22. Also, the hole 11 comprises an auxiliary recessed portion 24 extending inwards from the mid portion 23 along the length of the second interlocking means 7, away from the passage 22 and partly towards the second snap-locking means 13, the width of the auxiliary recessed portion 25 being less than the diameter of the mid portion 23.

In other embodiments, the stem 21 and shanks 16, 17 could switch positions, such that the shanks 16, 17 are provided on the sample collection body and the stem 21 on the first body.

The recesses of the sample collection body should be able to absorb mucous fluid and cells therein, and maintain these materials in place during retraction of the self-sampling device 1. In the present embodiment, the size of the sample collection body is slightly bigger than the one described in EP1903946B1, in order to allow for collection of more sample material and hold it deeper in recesses of the sample collection body.

The first body and the sample collection body can be manufactured of any suitable materials as desired. For example, these components may be formed of the same or different plastic materials. In the first embodiment, the first body is made of a plastic material, such as polypropylene, with a flexural modulus giving the first body flexibility to follow the anatomy of the vagina to reach portio vaginalis and fornix vaginalis and at the same time rigid enough to get a close contact between the sample collection body and the mucous tissue ectocervix.

A suitable method of manufacturing the first body and the sample collection body is injection molding, since it provides good repeatability and low cost.

At use, the self-sampling device 1 is moved to its first position with the sample collection body attached to the first body/handle/shaft 8.

Thereafter, the sample collection body is inserted into the vaginal region and moved to collect a sample. Thereafter, the self-sampling device 1 is retracted and the sample collection body inserted in a sealable transportation container 26. Subsequently, the first body is rotated by one hand relative to the sample collection body to the second position.

The sample collection body is prevented from rotating by the container which is held by the other hand. By keeping the container upright, the sample collection body is free to fall deeper into the container.

1. A self-sampling device for vaginal fluid from a vagina of a user, said self-sampling device comprising:
an elongate first body for insertion into the vagina of the user, and
a sample collection body for collecting a vaginal fluid sample from the user,
wherein the sample collection body is attachable to an inner end portion of the elongate first body by a releasable connection means,
wherein the elongate first body is configured such that it is operable by the user to collect a fluid sample from the vagina with the sample collection body attached to the first body,
wherein said releasable connection means comprises a first interlocking means provided on one of the first body and the sample collection body, and a second interlocking means provided on the other one of the elongate first body and the sample collection body,
wherein the first interlocking means is provided with a shaft defining a rotational axis substantially transversal to a longitudinal axis of the first body,
wherein the second interlocking means comprises a first hole or recess configured to receive the shaft upon connection of the first and second interlocking means for rotation of the sample collection body relative to the first body about the rotational axis between a first position, in which the longitudinal axis of the sample collection body is aligned with the longitudinal axis of the first body, and a second position in which the longitudinal axis of the sample collection body is rotated about the rotational axis away from said first position a predetermined distance,
wherein the first and second interlocking means are further provided with first and second respective corresponding snap-locking means configured to releasably engage each other when in the first position to prevent relative rotation between the first and second interlocking means when the snap-locking means are engaged,
wherein the first and second interlocking means are configured such that in said second position the first and second interlocking means are disengageable by movement of the shaft out of the first hole or recess in a predetermined disengagement direction, and
wherein the first and second interlocking means are configured such that in said first position the shaft is confined within said first hole or recess.

2. A self-sampling device according to claim 1, wherein the shaft is provided at a first proximal portion of the first interlocking means, wherein the first one of the corresponding snap-locking means is provided at a first distal portion of the first interlocking means.

3. A self-sampling device according to claim 2, wherein the first proximal portion of the first interlocking means is adjacent the first body or sample collection body on which the first interlocking means is provided, and wherein the first distal portion is further away from the sample collection body or first body on which the first interlocking means is provided than the first proximal portion.

4. A self-sampling device according to claim 1, wherein the first hole or recess is laterally open such that the shaft is moveable laterally out of the first hole or recess from said second position at disengagement.

5. A self-sampling device according to claim 1, wherein the first interlocking means comprises a forked body comprising opposite shanks together defining an intermediate space extending along the longitudinal axis of the first body,
wherein said shaft extends from one shanks to the other across said intermediate space, wherein the second interlocking means comprises an elongate stem adapted to fit in the intermediate space between the shanks, and
wherein said first hole or recess of the second interlocking means is laterally open for receiving the shaft.

6. A self-sampling device according to claim 5, wherein the shaft is provided at a first proximal portion of the first interlocking means, wherein a first one of the corresponding snap-locking means is provided at a first distal portion of the first interlocking means.

7. A self-sampling device according to claim 5, wherein the first hole or recess is laterally open in a direction along the length of the second interlocking means.

8. A self-sampling device according to claim 7,
wherein the shaft is mainly cylindrical with at least one recessed portion along the length of the shaft, and
wherein the first hole or recess has a cross sectional shape with a circular mid portion corresponding to the main diameter of the shaft,
wherein said mid portion opens laterally outwards in the form of a passage with a smallest width less than the diameter of the shaft, and wherein the recessed portion(s) of the shaft defines a projected profile of the shaft in a plane through the longitudinal axis of the shaft, said projected profile being smaller than the passage such that the shaft is movable through the passage from said second position.

9. A self-sampling device according to claim 8, wherein the passage widens outwardly away from the circular mid portion.

10. A self-sampling device according to claim 7, wherein the first hole or recess comprises an auxiliary recessed portion extending inwards from the circular mid portion along the length of the second interlocking means, away from the passage and partly towards the second snap-locking means, the width of the auxiliary recessed portion being less than the diameter of the mid portion.

11. A self-sampling device according to claim 7, wherein the corresponding snap-locking means are provided in the interface between stem and shanks.

12. A self-sampling device according to claim 11, wherein protrusions of the first snap-locking means are provided on the shanks wherein at least one recess of the second snap-locking means is provided in the stem, and wherein the protrusions of the first snap-locking means comprise forwardly chamfered portions oriented for the stem to gradually push the protrusions apart upon insertion of the stem between the chamfered portions.

13. A system comprising a self-sampling device according to claim 1, a sealable container for storing the sample collection body after use.
